(19) United States
(12) Patent Application Publication
(10) Pub. No.: US 2025/0120438 A1
(43) Pub. Date: Apr. 17, 2025

(54) A Portable Device and a Method of Manipulating a Portable Device

(71) Applicant: JT International SA, Geneva (CH)

(72) Inventor: Xavier Flavard, Paris (FR)

(73) Assignee: JT International SA, Geneva (CH)

(21) Appl. No.: 18/682,012

(22) PCT Filed: Aug. 4, 2022
(86) PCT No.: PCT/EP2022/071998

§ 371 Date: Feb. 7, 2024

(30) Foreign Application Priority Data
Aug. 12, 2021 (EP) 21191082.3

(51) Int. Cl. A24F 40/40; A24F 40/10

(52) U.S. Cl. CPC A24F 40/40; A24F 40/10

(57) ABSTRACT

A portable device includes a first part includes a mouthpiece, a second part pivotably connected to the first part about a first axis, and a third part pivotally connected to the first part about a second axis. The second axis is substantially perpendicular to the first axis. The portable device is adapted to be manipulated by a user between a non-use configuration where the mouthpiece is substantially surrounded by the side panels of the second and third parts, and a use configuration where the mouthpiece is exposed at an end of the portable device.

### TECHNICAL FIELD

The present disclosure relates to a portable device, and more particularly to a portable device such as an aerosol-generating device or vapour-generating device that may be manipulated manually by a user between a non-use configuration where a mouthpiece of the device is shielded, and a use configuration where the mouthpiece is exposed at an end of the device and may be used by the user to inhale an aerosol or vapour.

### TECHNICAL BACKGROUND

Devices which heat, rather than burn, an aerosol generating material to produce an aerosol or vapour for inhalation have become popular with consumers in recent years.

Such devices normally include a mouthpiece through which the aerosol or vapour is inhaled by a user. Because the mouthpiece is placed in the mouth of the user, there is a desire to keep the mouthpiece clean and to prevent contact with the mouthpiece when the device is not being used.

### SUMMARY OF THE DISCLOSURE

According to a first aspect of the present disclosure, there is provided a portable device comprising:
a first part (or main body) comprising a mouthpiece;
a second part (or first housing part) comprising first and second side panels having a first end pivotably connected to the first part about a first axis; and
a third part (or second housing part) comprising third and fourth side panels having a first end pivotally connected to the first part about a second axis, the second axis being substantially perpendicular to the first axis;
wherein the portable device is adapted to be manipulated by a user between a non-use configuration where the mouthpiece is substantially surrounded by the first, second, third and fourth side panels, and a use configuration where the mouthpiece is exposed at an end of the portable device.

When the device is in the non-use configuration, the mouthpiece is shielded because it is positioned in a substantially enclosed volume defined by the side panels of the second and third parts. This keeps the mouthpiece clean and prevents contact with the mouthpiece when the device is not being used.

When the device is in the use configuration, the user may use the exposed mouthpiece to inhale an aerosol or vapour generated by the device while grasping the second and third parts.

In general terms, a vapour is a substance in the gas phase at a temperature lower than its critical temperature, which means that the vapour can be condensed to a liquid by increasing its pressure without reducing the temperature, whereas an aerosol is a suspension of fine solid particles or liquid droplets, in air or another gas. It should, however, be noted that the terms ‘aerosol’ and ‘vapour’ may be used interchangeably in this specification, particularly with regard to the form of the inhalable medium that is generated for inhalation by a user.

The device will preferably comprise additional components. For example, if the device is an aerosol-generating device or a vapour-generating device, the device will preferably comprise additional components such as a heating system for heating an aerosol or vapour generating material, a controller, a printed circuit board (PCB), a battery etc. Such additional components may be provided in one or more of the first, second and third parts. One or both of the second and third parts may be electrically connected to the first part to permit the additional components to be distributed between the parts of the device. The electrical connection may be provided using suitable connectors that also permit the second and third parts to be pivotally connected to the first part. The second and third parts of the device may be formed as solid pieces from one or more materials for reasons of manufacturing and cost efficiency. As such, the additional components would be provided in the first part of the device. The second and third parts may alternatively be formed as hollow pieces or housings inside which one or more of the additional components may be provided. For example, one of the second and third parts may be provided with a battery or a PCB. This allows the first part to be smaller in size and more physically compact because it does not have to contain all of the additional components. The second part may include a battery and the third part may include a PCB or vice versa. The battery may be substantially u-shaped and may extend along the side panels of the second part or third part. The first end of the side panels may include electrical connectors of different polarity such as a first electrical connector of positive polarity and a second electrical connector of negative polarity that may be electrically connected to corresponding battery terminals. One of the side panels of the second part or third part may include a first PCB and the other side panel may include a second PCB. The first and second PCBs may be electrically connected together. The first end of the side panels may include electrical connectors for the PCB(s). The electrical connectors may have different polarities such as a first electrical connector of positive polarity and a second electrical connector of negative polarity that may be electrically connected to corresponding PCB terminals. The electrical connectors may allow the battery to be electrically connected to the PCB(s) and to any other additional components provided in the first part of the device.

The first part may be pivotally connected to the second part by a first connector. The first part may be pivotally connected to the third part by a second connector. The first and second connectors may be designed to provide an electrical connection if necessary. The first connector may be configured to selectively lock the second part relative to the first part in a particular configuration. The second part may only be free to pivot relative to the first part if the first connector is unlocked by the user. For example, the second part may be allowed to pivot only if part of the first connector (e.g., a protrusion with a key provided on the first part) is disengaged from another part (e.g., an opening with a keyway provided on the second part) by the user applying a force to the second part that moves the first and second side panels closer together against a bias force. When the part of the first connecter is not disengaged from the other part, the second part is locked relative to the first part. The second connector may similarly be configured to lock the third part relative to the first part in a particular configuration. The third part may only be free to pivot relative to the first part if the second connector is unlocked by the user. For example, the third part may be allowed to pivot only if part of the second connector (e.g., a protrusion with a key provided on the first part) is disengaged from another part (e.g., an opening with a keyway provided on the third part) by the user applying a force to the third part that moves the third and fourth side panels closer together against a bias force. When the part of the second connector is not disengaged from the other part, the third part is locked relative to the first part. Selectively locking the device in a particular configuration, such as the non-use configuration, helps to prevent the device from being used by a child (who might be expected not to be able to selectively unlock the locked connector(s)), or from being accidentally manipulated away from the non-use configuration.

The second part may be substantially u-shaped and may further comprise an end panel connected between the first and second side panels at a second end of the first and second side panels. If the second part is substantially u-shaped it may have a natural bias force or spring effect against which the user may apply a force to unlock the first connector. This bias force will normally be sufficient to ensure that a locked connector is only unlocked if the user deliberately presses the first and second side panels together.

The third part may be substantially u-shaped and may further comprise an end panel connected between the third and fourth side panels at a second end of the third and fourth side panels. If the third part is substantially u-shaped it may have a natural bias force or spring effect against which the user may apply a force to unlock the second connector. The bias force will normally be sufficient to ensure that a locked connector is only unlocked if the user deliberate presses the third and fourth side panels together.

The end panels of the second and third parts may be positioned adjacent to each other when the device is in the non-use and use configurations.

In the non-use configuration, the end panel of the second part may be positioned outside the end panel of the third part, and in the use configuration, the end panel of the third part may be positioned outside the end panel of the second part. In general terms, in the non-use configuration, the third part is nested within the second part, and in the use configuration, the second part is nested within the third part.

The first and second side panels may be substantially parallel and the third and fourth side panels may be substantially parallel. The first and second side panels may be substantially perpendicular to the third and fourth side panels when the portable device is in the non-use and use configurations.

The mouthpiece may be provided at a first end of the first part. The first part may further comprise a connector receptacle at a second end of the first part opposite the first end. The connector receptacle may be a USB-type receptacle or a mating receptacle for a ‘pogo’ pin or spring-loaded pin, for example. In the use configuration, the connector receptacle is shielded because it is positioned in a substantially enclosed volume defined by the side panels of the second and third parts. In the non-use configuration, when the mouthpiece is shielded, the connector receptacle is exposed at an end of the device. The connector receptacle may receive any suitable connector and may be used for charging the device and/or data communication, for example. Because the mouthpiece is shielded, it means that the user cannot use the device (e.g., to generate an aerosol or vapour) when the device is being charged through the connector receptacle when the connector receptable is exposed at an end of the device in the non-use configuration. This provides a safety improvement because battery regulations typically recommend that the device is not used while it is being charged.

According to a second aspect of the present disclosure, there is provided a method of manipulating a portable device according to any preceding claim, comprising:
from the non-use configuration, pivoting together the first and third parts by substantially 180 degrees relative to the second part about the first axis to an intermediate configuration, and
from the intermediate configuration, pivoting the third part by substantially 180 degrees relative to the first and second parts about the second axis to the use configuration.

The method may be carried out in reverse to manipulate the device from the use configuration to the non-use configuration.

The user may manually manipulate the device easily and intuitively between the non-use and use configurations. An important benefit of the disclosure is that when the device is in the non-use configuration, the mouthpiece is shielded, and when the device is in the use configuration, the mouthpiece is exposed at an end of the device and the second and third parts can be grasped by the user.

### BRIEF DESCRIPTION OF THE DRAWINGS

FIGS. 1, 2 and 3 are diagrammatic perspective, front end and side views respectively of a main body forming part of an example of an aerosol-generating device or vapour generating-device:

FIG. 4 is a diagrammatic perspective view of a first housing part forming part of the example of the aerosol-generating device or vapour-generating device:

FIG. 5 is a diagrammatic perspective view of a second housing part forming part of the example of the aerosol-generating device or vapour-generating device:

FIGS. 6 to 10 are diagrammatic perspective views showing how the example of the aerosol-generating device or vapour-generating device is manipulated from a non-use configuration to a use configuration;

FIG. 11 is a diagrammatic perspective view showing a battery located in a first housing part forming part of the example of the aerosol-generating device or vapour-generating device:

FIG. 12 is a diagrammatic perspective view showing printed circuit boards (PCBs) located in a second housing part forming part of the example of the aerosol-generating device or vapour-generating device:

FIG. 13 is a diagrammatic perspective view showing another example of an aerosol-generating device or vapour-generating device:

FIGS. 14 and 15 are diagrammatic side and end views respectively of a protrusion forming part of the another example of the aerosol-generating device or vapour-generating device; and

FIGS. 16 and 17 are diagrammatic cross-section views showing how the protrusion is received in an opening of the another example of the aerosol-generating device or vapour-generating device.

### DETAILED DESCRIPTION OF EMBODIMENTS

Embodiments of the present disclosure will now be described by way of example only and with reference to the accompanying drawings.

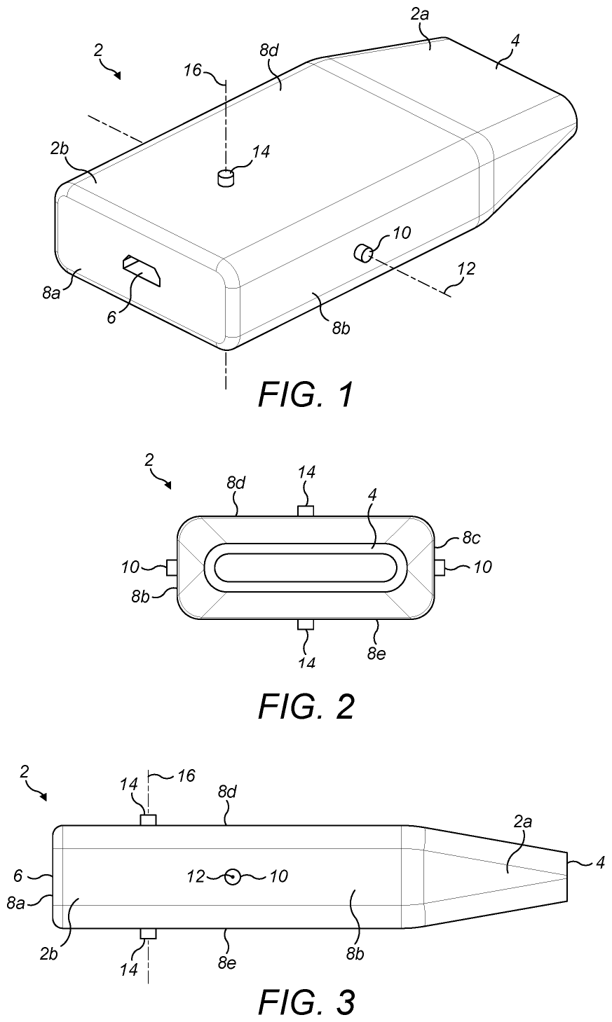

Initially referring to FIGS. 1 to 3, there is shown a main body 2 forming part of an example of a portable device 1 for generating an aerosol or vapour. The main body 2 includes a mouthpiece 4 at a first end 2a through which an aerosol or vapour generated by the device may be inhaled by a user. A connector receptacle 6 such as a USB-type receptacle is provided in an end surface 8a at a second end 2b of the main body 2 for charging the device and/or data communication, for example. Other components for generating the aerosol or vapour and/or for controlling the operation of the device 1 may also be included in the main body 2.

The main body 2 includes opposite side surfaces 8b and 8c, an upper surface 8d and a lower surface 8e.

The side surfaces 8b and 8c include pivot locations 10 which define a first axis 12. The upper and lower surfaces 8d and 8e include pivot locations 14 which define a second axis 16. The first and second axes 12 and 16 are substantially perpendicular to each other. The main body 2 defines a mutually orthogonal coordinate system comprising a longitudinal axis, a lateral axis, and a transverse axis. The first axis 12 is substantially parallel to a transverse axis of the main body 2 and the second axis 16 is substantially parallel to a lateral axis of the main body. The first and second axes 12 and 16 are offset and spaced apart in the longitudinal axis. In the illustrated example, the first axis 12 is closer to the mouthpiece 4 than the second axis 16.

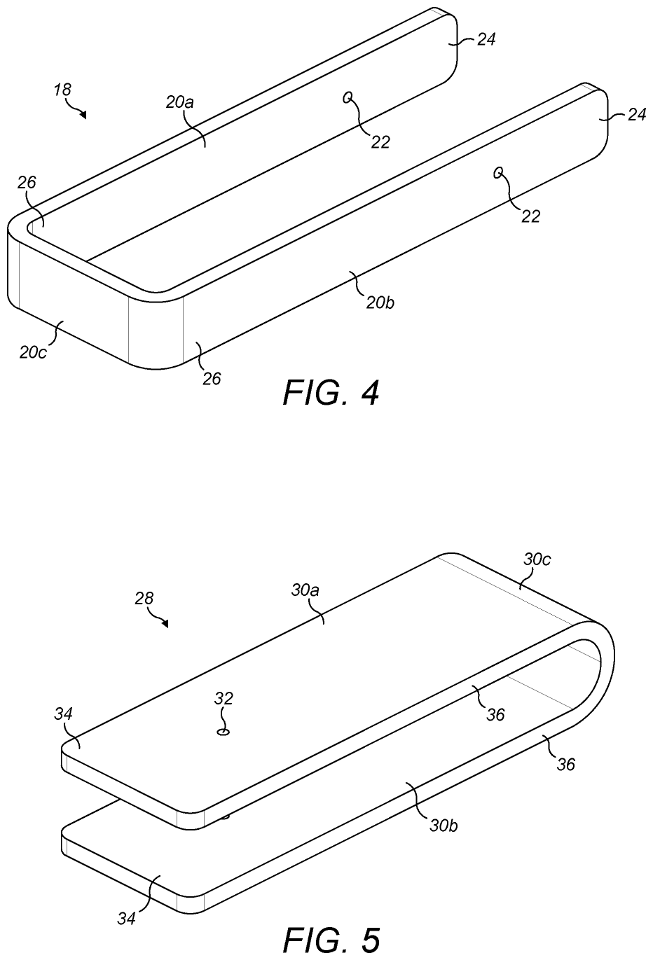

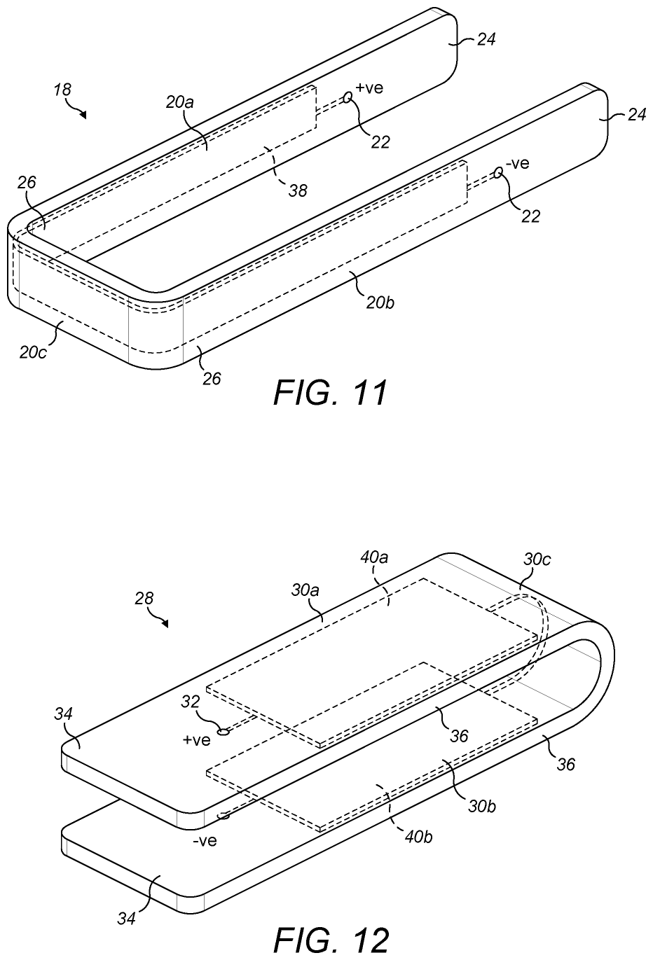

A first housing part 18 is shown in FIG. 4. The first housing part 18 is substantially u-shaped and includes a pair of substantially parallel side panels 20a and 20b and an end panel 20c. Each side panel 20a and 20b includes a pivot location 22 at a first end 24. The end panel 20c is connected between the side panels 20a and 20b at a second end 26. FIG. 11 shows a battery 38 positioned within the first housing part 18. The battery 38 has a positive terminal that is electrically connected to a positive connector at the pivot location 22 on the side panel 20a and a negative terminal that is electrically connected to a negative connector at the pivot location 22 on the side panel 20b.

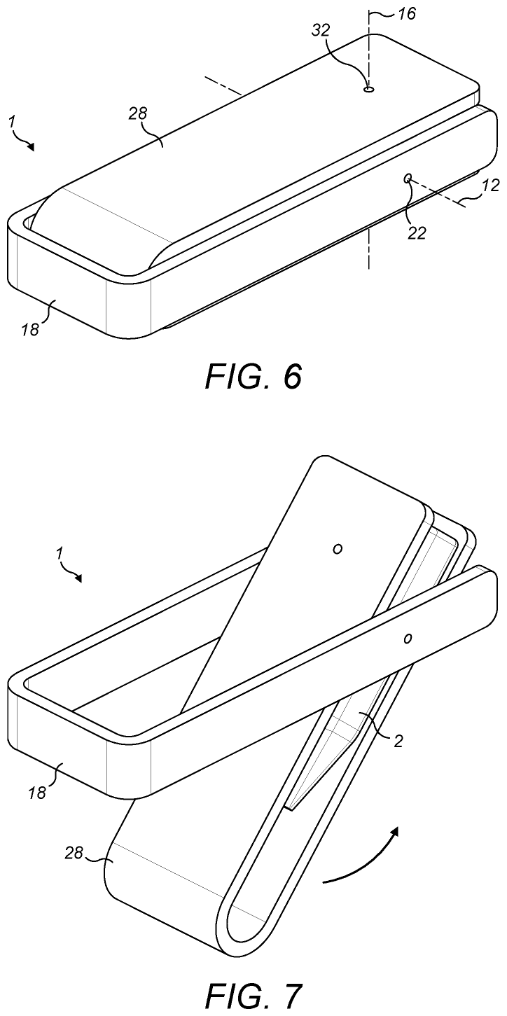

A second housing part 28 is shown in FIG. 5. The second housing part 28 is substantially u-shaped and includes a pair of substantially parallel side panels 30a and 30b and an end panel 30c. Each side panel 30a and 30b includes a pivot location 32 at a first end 34. The end panel 30c is connected between the side panels 30a and 30b at a second end 36. FIG. 12 shows two printed circuit boards (PCBs) 40a and 40b positioned within the second housing part 28. The PCBs 40a and 40b are electrically connected together as shown. The PCB 40a has a positive terminal that is electrically connected to a positive connector at the pivot location 32 on the side panel 30a and the PCB 40b has a negative terminal that is electrically connected to a negative connector at the pivot location 32 on the side panel 30b.

The first and second housing parts 18 and 28 are shaped and sized to permit the user manipulation described below.

The assembled device 1 is shown in FIG. 6 in a non-use configuration. The first housing part 18 is pivotably connected to the main body 2 about the first axis 12 by means of the pivot locations 10 and 22. The pivot locations 10 and 22 are defined by connectors that allow the first housing part 18 to pivot relative to the main body 2. In the illustrated example, the connectors at pivot locations 10 are shown as protrusions that extend outwardly from the side surfaces 8b and 8c of the main body 2 and the connectors at pivot locations 22 are shown as openings that receive the protrusions. But it will be readily understood that other connectors are possible, including connectors such as those shown in FIG. 11 that also provide an electrical connection between the main body and the first housing part. In another example, the connectors at the pivot locations on the first housing part can be protrusions and the connectors at the pivot locations on the main body can be indentations that receive the protrusions. In yet another example, the connectors can be defined by pivot axles or pins.

The second housing part 28 is pivotably connected to the main body 2 about the second axis 16 by means of the pivot locations 14 and 32. The pivot locations 14 and 32 are defined by connectors that allow the second housing part 28 to pivot relative to the main body 2. In the illustrated example, the connectors at pivot locations 14 are shown as protrusions that extend outwardly from the upper and lower surfaces 8d and 8e of the main body 2 and the connectors at pivot locations 32 are shown as openings that receive the protrusions. But it will be readily understood that other connectors are possible, including connectors such as those shown in FIG. 12 that also provide an electrical connection between the main body and the second housing part. In another example, the connectors at the pivot locations on the second housing part can be protrusions and the connectors at the pivot locations on the main body can be indentations that receive the protrusions. In yet another example, the connectors can be defined by pivot axles or pins.

In the non-use configuration, the mouthpiece 4 is substantially surrounded by the side panels 20a, 20b, 30a and 30b which together define a substantially enclosed volume in which the mouthpiece is positioned. The side panels shield the mouthpiece 4. This keeps the mouthpiece 4 clean and prevents contact with the mouthpiece when the device is not being used. The connector receptacle 6 is exposed at an end of the device 1 for receiving a connector such as a USB-type connector for charging and/or data communication.

The end panel 20c of the first housing part 18 is positioned adjacent and outside of the end panel 30c of the second housing part 28 as shown in FIG. 6. The second housing part 28 is prevented from pivoting relative to the main body 2 about the second axis 16 by the side panels 20a and 20b of the first housing part 18.

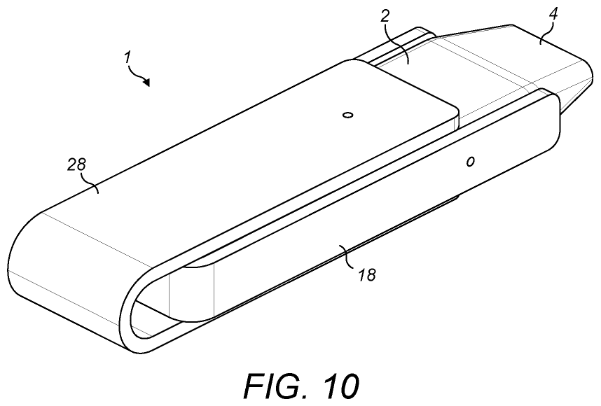

The device 1 is adapted to be manipulated by a user between the non-use configuration shown in FIG. 6 and a use configuration shown in FIG. 10 where the mouthpiece 4 is exposed at an end of the device.

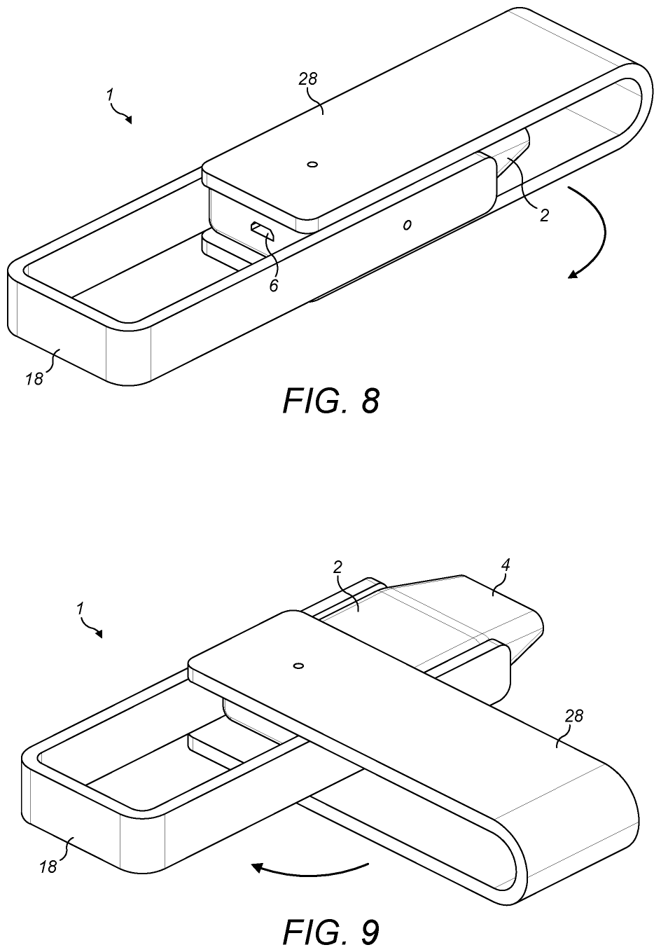

In a first step, the main body 2 and the second housing part 28 are pivoted together by substantially 180 degrees relative to the first housing part 18 about the first axis 12 (see FIG. 7) such that the device 1 is in an intermediate configuration shown in FIG. 8. For example, this may be done by pushing the second housing part 28 and the main body 2 out of the plane of the first housing part 18 and pivoting them around to the intermediate configuration by manipulating one or both of the first and second housing parts. The second housing part 28 and the main body 2 pass freely and without obstruction through the volume defined by the side panels 20a and 20b, and the end panel 20c, of the first housing part 18.

In the intermediate configuration shown in FIG. 8, the first and second housing parts 18 and 28 are arranged in the same plane at opposite ends of the device 1.

In a second step, the second housing part 28 is pivoted by substantially 180 degrees relative to the first housing part 18 and the main body 2 about the second axis 16 (see FIG. 9) such that the device 1 is in the use configuration shown in FIG. 10. During the second step, main body 2 and the first and second housing parts 18 and 28 remain in the same plane and the second housing part 28 passes over the side panels 20a and 20b of the first housing part 18. Put another way, the first housing part 18 passes freely and without obstruction through the volume defined by the side panels 30a and 30b, and the end panel 30c, of the second housing part 28. The first and second pivot locations 10 and 14 are offset in the longitudinal axis of the main body 2 to permit the end panel 30c of the second housing part 28 to avoid coming into contact with the end panel 20c of the first housing part 18 as it is pivoted towards the use configuration. In the use configuration, the mouthpiece 4 is exposed at an end of the device 1 and the end panel 30c of the second housing part 28 is positioned adjacent and outside of the end panel 20c of the first housing part 18 as shown in FIG. 10. The first and second housing parts 18 and 28 may be grasped by the user when using the device 1, and in particular can be used as a handle when inhaling an aerosol or vapour from the mouthpiece 4.

The steps may be carried out in reverse to manipulate the device 1 from the use configuration to the non-use configuration where the mouthpiece 4 is shielded.

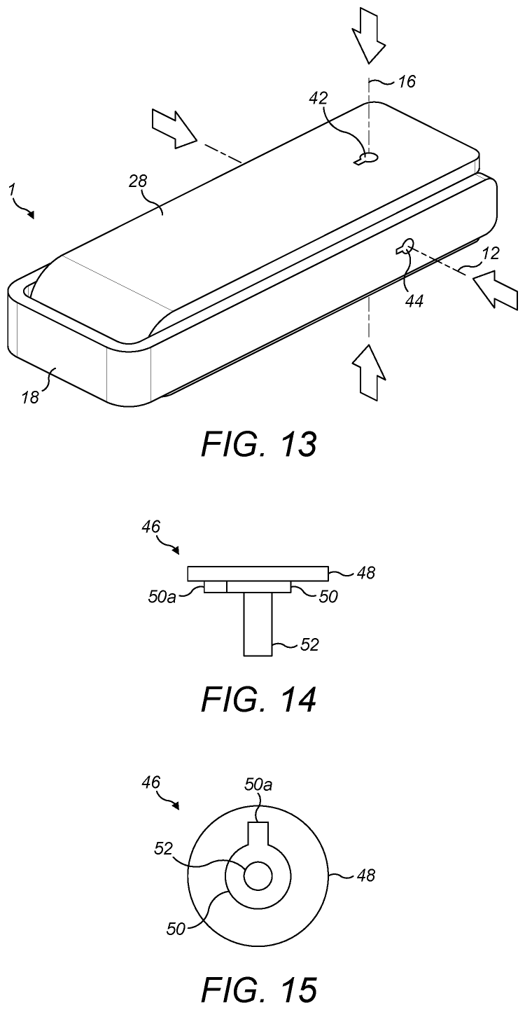

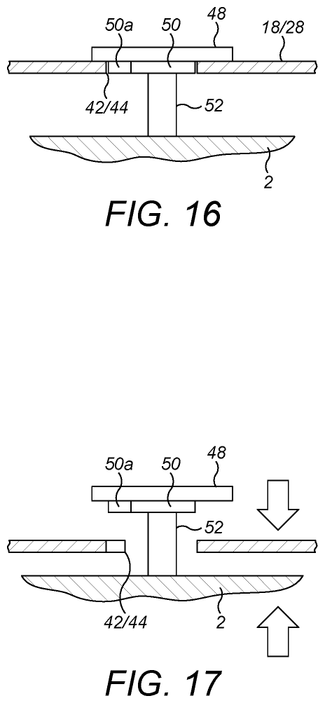

FIG. 13 shows a device where the first and second housing parts 18 and 28 may be selectively locked relative to the main body 2 in the non-use configuration. An opening 42 in the first housing part 18 that defines a connector at the pivot location 22 is formed with a keyway and an opening 44 in the second housing part 28 that defines a connector at the pivot location 32 is formed with a keyway. FIGS. 14 and 15 show a protrusion 46 that is formed on the main body 2 and is constructed to be received in one of the openings 42 and 44 in the respective housing part. The protrusion 46 includes a head part 48 that is larger than the opening in the housing part, a locking part 50 that is shaped to be complementary to the opening and which includes a radially-extending key 50a, and a shaft part 52. FIG. 16 shows how, when the user is not applying a force to the side panels of the housing part, the locking part 50 is positioned in the opening with the key 50a in the keyway so as to prevent relative rotation between the main body 2 and the respective housing part. However, if the user applies a force to the side panels of the housing part by pressing them together at the first end against the bias force that results from the fact that the housing part is substantially u-shaped, which user-applied force is indicated in FIGS. 13 and 17 by the opposing pairs of block arrows, it can be seen in FIG. 17 that the locking part 50 is disengaged from the opening. The opening is now aligned with the shaft part 52 and the housing part is able to pivot relative to the main body 2. The device can be selectively locked in the non-use configuration and only permitted to be manipulated to the use configuration if the user first unlocks the first and second housing parts 18 and 28 from the main body 2 by applying a force to the respective side panels. In practice, the user may unlock the first housing part 18 from the main body 2 to allow the main body 2 and the second housing part 28 to be pivoted together by substantially 180 degrees relative to the first housing part 18 about the first axis 12 such that the device 1 is in an intermediate configuration shown in FIG. 8. The user may then unlock the second housing part 28 from the main body 2 to allow the second housing part to be pivoted by substantially 180 degrees relative to the first housing part 18 and the main body 2 about the second axis 16 such that the device 1 is in the use configuration shown in FIG. 10. When the device is manipulated in reverse, the radially-extending key of the locking part 50 will become aligned with the keyway of the respective opening. The bias force of the housing part, which pushes the side panels apart, will reposition the locking part in the opening to lock the housing part to the main body 2 and prevent relative rotation.

Although exemplary embodiments have been described in the preceding paragraphs, it should be understood that various modifications may be made to those embodiments without departing from the scope of the appended claims. Thus, the breadth and scope of the claims should not be limited to the above-described exemplary embodiments.

Any combination of the above-described features in all possible variations thereof is encompassed by the present disclosure unless otherwise indicated herein or otherwise clearly contradicted by context.

Unless the context clearly requires otherwise, throughout the description and the claims, the words “comprise”, “comprising”, and the like, are to be construed in an inclusive as opposed to an exclusive or exhaustive sense; that is to say, in the sense of “including, but not limited to”.

1. A portable device comprising:
a first part comprising a mouthpiece;
a second part comprising first and second side panels having a first end pivotably connected to the first part about a first axis; and
a third part comprising third and fourth side panels having a first end pivotally connected to the first part about a second axis, the second axis being substantially perpendicular to the first axis;
wherein the portable device is adapted to be manipulated by a user between a non-use configuration where the mouthpiece is substantially surrounded by the first, second, third and fourth side panels, and a use configuration where the mouthpiece is exposed at an end of the portable device.

2. The portable device according to claim 1, wherein the second part is substantially u-shaped and further comprises an end panel connected between the first and second side panels at a second end of the first and second side panels.

3. The portable device according to claim 1, wherein the third part is substantially u-shaped and further comprises an end panel connected between the third and fourth side panels at a second end of the third and fourth side panels.

4. The portable device according to claim 2, wherein the third part is substantially u-shaped and further comprises an end panel connected between the third and fourth side panels at a second end of the third and fourth side panels, and wherein the end panels of the second and third parts are positioned adjacent to each other when the portable device is in the non-use and use configurations.

5. The portable device according to claim 4, wherein in the non-use configuration the end panel of the second part is positioned outside the end panel of the third part, and in the use configuration the end panel of the third part is positioned outside the end panel of the second part.

6. The portable device according to claim 1, wherein the first and second side panels are substantially parallel and the third and fourth side panels are substantially parallel.

7. The portable device according to claim 6, wherein the first and second side panels are substantially perpendicular to the third and fourth side panels when the portable device is in the non-use and use configurations.

8. The portable device according to claim 1, wherein the mouthpiece is provided at a first end of the first part, and wherein the first part further comprises a connector receptacle at a second end opposite the first end.

9. The portable device according to claim 1, wherein the main body defines a mutually orthogonal coordinate system comprising a longitudinal axis, a lateral axis, and a transverse axis, wherein the first axis is substantially parallel with the transverse axis and the second axis is substantially parallel with the lateral axis.

10. The portable device according to claim 9, wherein the first and second axes are spaced apart in the longitudinal axis.

11. The portable device according to claim 1, being wherein the portable device is a device for generating an aerosol or vapour that may be inhaled by a user through the mouthpiece.

12. The portable device according to claim 1, wherein one of the second part and the third part further comprises a battery and other one of the second part and the third part further comprises a printed circuit board.

13. The portable device according to claim 1, wherein the first part is pivotally connected to the second part or the third part by a connector.

14. The portable device according to claim 13, wherein the connector is configured to selectively lock the second part or the third part relative to the first part in the non-use configuration.

15. The method of manipulating a portable device according to claim 1, comprising:
from the non-use configuration, pivoting together the first and third parts by substantially 180 degrees relative to the second part about the first axis to an intermediate configuration, and
from the intermediate configuration, pivoting the third part by substantially 180 degrees relative to the first and second parts about the second axis to the use configuration.

16. The portable device according to claim 13, wherein the connector is configured to provide an electrical connection.
